(19) United States
(12) Patent Application Publication
(10) Pub. No.: US 2023/0204552 A1
(43) Pub. Date: Jun. 29, 2023

(54) METHOD FOR THE TEMPERATURE-DEPENDENT DETECTION OF GAS USING A GAS-SELECTIVE MEMBRANE

(71) Applicant: INFICON GMBH, Köln (DE)

(72) Inventor: Silvio DECKER, Köln (DE)

(73) Assignee: INFICON GMBH, Köln (DE)

(21) Appl. No.: 18/009,584

(22) PCT Filed: May 25, 2021
(86) PCT No.: PCT/EP2021/063924

§ 371 Date: Dec. 9, 2022

(30) Foreign Application Priority Data
Jun. 24, 2020 (DE) 10 2020 116 660.5

(51) Int. Cl. G01N 33/00

(52) U.S. Cl. CPC G01N 33/0016

(57) ABSTRACT

In a method for the detection of gas using a gas-selective membrane, a temperature device that is designed to change the temperature of the membrane, and a detector that is designed to acquire a measurement signal on the basis of the amount of gas passing through the membrane, provision is made for the following steps: changing the temperature of the membrane using the temperature device, acquiring at least one first measuring value (Hn, Hn+1, Hn+2) using the detector at a time at which the membrane temperature adopts a first temperature value, acquiring at least one second measuring value (Ln, Ln+1) using the detector at a time at which the membrane temperature adopts a second temperature value different from the first temperature value, calculating the difference between the two measuring values, and using the difference to assess whether a gas to be detected is present.

The invention relates to a method for gas detection using a gas-selective membrane, the permeability of which to a specific gas is dependent on temperature.

For gas detection using such gas-selective membranes, it is known to set the membrane temperature to a temperature value at which the gas permeability of the membrane to the gas type to be detected is maximal. For this purpose, the membrane is usually heated, when the gas permeability is maximal at a temperature that is higher than room temperature.

The amount of gas permeating through the membrane depends on the temperature of the membrane and on the partial pressure difference of the gas upstream, i.e. in front of the membrane and of the gas downstream, i.e. behind the membrane. The detection of the gas to be detected downstream of the membrane is performed using a detector which may be a gas measuring device, e.g. a mass spectrometer, or a pressure measuring device measuring the total pressure of the gas. A total pressure measurement of the gas is advantageous, if the membrane is highly selective to a specific type of gas.

In the temperature-dependent gas detection using a gas selective membrane, the measuring signal of the gas is superposed by an offset signal which is dependent, for example, on the temperature or on the age of the gas sensor. In order to determine the offset signal, the gas can be removed upstream of the sensor by evacuation using pumps, so as to measure the offset signal in a vacuum without gas.

It is an object of the invention to provide a method for the detection of gas using a gas-selective membrane in which the influence of the offset signal on the measuring signal is reduced.

The method according to the invention is defined by the features of claim 1.

The das detection according to the invention is performed using a gas-selective membrane with a temperature-dependent permeability to the gas to be detected. A temperature device is configured to change the temperature of the membrane. The temperature device preferably is a heating. However, a cooling device is also conceivable, if the permeability of the membrane is maximal at a temperature lower than the ambient temperature of the membrane. A detector is provided and configured to detect a measuring signal in dependence on the amount of the gas passing through the membrane. The detector may be a gas measuring device, e.g. a mass spectrometer, or a pressure measuring device, e.g. a total pressure measuring device.

According to the method of the invention, the temperature of the membrane is changed using the temperature device so as to sequentially adjust the membrane temperature in chronological order to at least two different values different from zero. At least one first measuring value at a time at which the membrane temperature adopts a first temperature value is acquired from the measuring signal of the detector. Thereafter, at least a second measuring value of the measuring signal at a second time different from the first time is acquired, the membrane temperature at the second time adopting a second temperature value different from the first temperature value. The difference is calculated from the two measuring values acquired. The assessment, whether a gas to be detected is present and has been measured, is made based on the calculated differential signal.

The permeability of the gas-selective membrane to the gas to be detected should be higher at one of the two membrane temperatures than at the other membrane temperature. For example, the permeability may be higher at the first temperature value of the membrane temperature than at the second temperature value. Preferably, the permeability is maximal at the first temperature value.

If the permeability of the membrane is lower at the second membrane temperature than at the first membrane temperature, the proportion of the offset is less in the second measuring value than in the first measuring value. On the other hand, the proportion of the measuring signal that is caused by the gas to be detected due to the increased permeability is higher in the first measuring value. The proportion of the offset is reduced by the calculation of the difference.

Preferably, the membrane temperature is changed periodically such that the membrane temperature alternately adopts the two temperature values sequentially at periodically recurring intervals, the measuring values for the respective temperatures being acquired at at least two different intervals. Thus, the first measuring value is acquired in successive intervals in each case, when the membrane temperature adopts the first temperature value. Accordingly, the second measuring value is acquired in successive intervals in each case, when the membrane temperature adopts the second temperature value.

The change in membrane temperature can be performed by means of a control between two temperature values different from zero. The temperature control can be performed by measuring the membrane temperature using a temperature measuring device and by heating the membrane using a heating device, in order to control the temperature of the membrane from a lower temperature value to a higher temperature value.

When calculating the difference for the two measuring values of subsequent intervals, mean values of the measuring values can be calculated and used over subsequent intervals in each case. For example, the difference can be calculated between the mean value of the first measuring values of at least two different intervals and the second measuring values between these two first measuring values. As an alternative, the difference can also be calculated between the mean value of at least two different intervals and the first measuring value between the two second measuring values.

In order to change the temperature of the membrane, a parameter which influences the membrane temperature can be adjusted and changed at the temperature device. The change of this parameter and the resultant change of the membrane temperature from one temperature value to the next temperature value should occur only after a period of at least 2 seconds and preferably a period in the range from approx. 5 to 15 seconds therebetween.

The difference between the different temperature values of the membrane temperature, which are adjusted and changed by means of the temperature device, should be between approx. 2 K and 10 K and preferably between approx. 3 K and 6 K. This applies in particular to the difference between the first temperature value and the second temperature value. Here, the temperature device is preferably designed as a heating, with at least the first temperature value of the membrane temperature being higher than the temperature of the membrane environment. Prior to adjusting a subsequent new temperature value of the membrane temperature, the heating can be turned off for a few seconds, the set first temperature value and the set second temperature value of the membrane temperature each being higher than the ambient temperature. The measuring values of the measuring signals are advantageously acquired in each relevant interval only after several seconds, preferably after about 2 to 5 seconds, after a new temperature value has been set at the temperature device or the parameter influencing the membrane temperature has been changed.

In the method of the invention, preferably no pump is used to generate a differential pressure between the pressure of the gas upstream of the membrane and the pressure of the gas downstream of the membrane. The method of the invention can be used, in particular, for the detection of a gas in a room of a building.

An embodiment of the invention will be explained in detail hereunder with reference to the Figures. In the drawings:

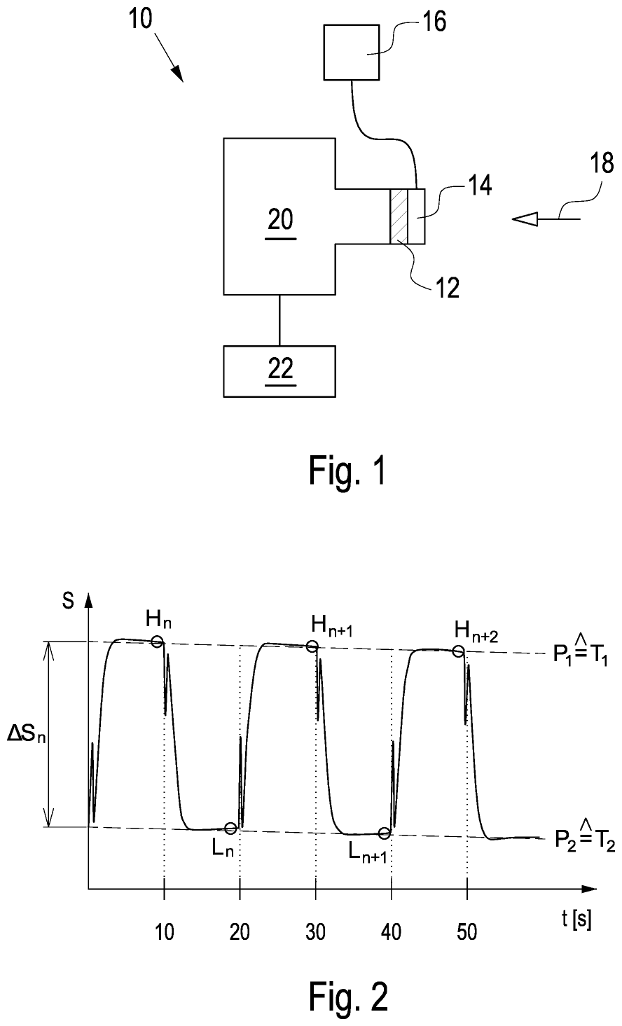

FIG. 1 is a schematic illustration of the gas detector,

FIG. 2 is a schematic illustration of the measuring signal and

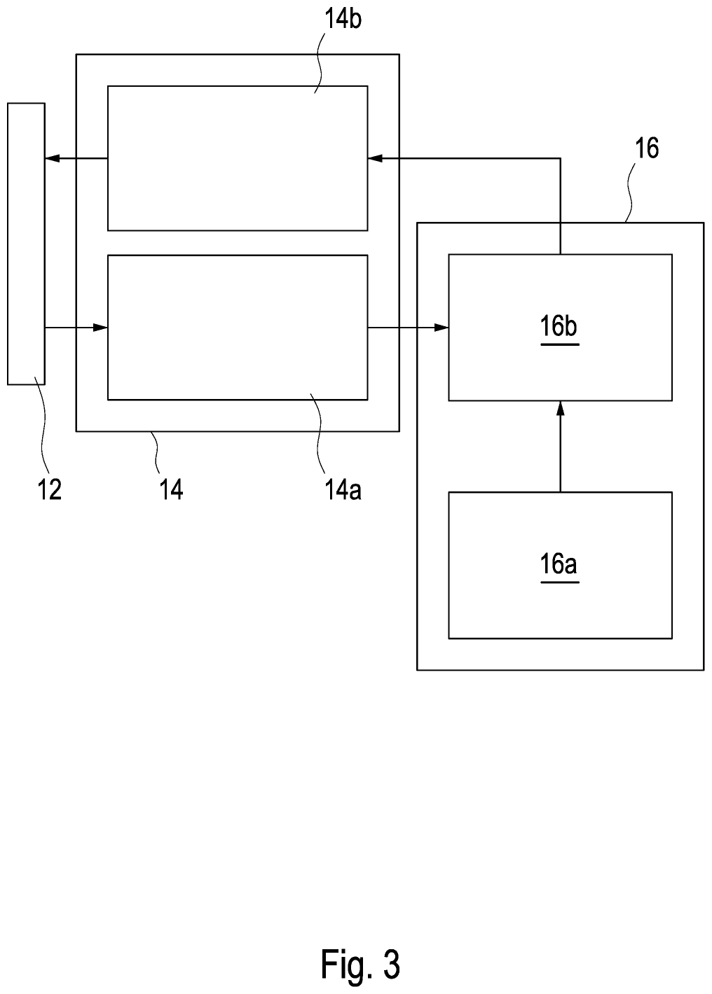

FIG. 3 is a schematic illustration of the temperature and control devices in FIG. 1.

FIG. 1 schematically illustrates the gas detection device 10 having a gas-selective membrane 12 with temperature-dependent permeability. The membrane 12 is heated by a temperature device 14 in the form of a heating with heating elements arranged on the membrane 12. The heating device is controlled via a control device 16. The control device 16 is part of the temperature device 14. Parameters that influence the output of the heating and thus the heating effect of the membrane 12 are inputted via the control device 16. Thereby, a parameter that influences the heating 14 and changes the temperature of the membrane 12 can be changed via the control device 16. The heating 14 is installed at the membrane 12 such that gas can pass through the heating 14 and through the membrane 12 in the direction of the arrow 18 in FIG. 1. A detector 20 in the form of a pressure measuring device is arranged downstream, i.e. behind the membrane 12. with respect to the flow direction according to the arrow 18 in FIG. 1. Gas permeating through the membrane 12 arrives in the detector and increases the pressure measured there. As an alternative, the detector 20 may also be a gas detector, e.g. in the form of a mass spectrometer.

It can be seen in FIG. 3 that the temperature device 14 comprises a temperature sensor 14a for measuring the membrane temperature and a membrane heating 14b for heating the membrane 12 and that the control device 16 comprises a temperature presetting means 16 for inputting the membrane temperature to be adjusted via the membrane heating 14b and a control logic 16b which controls the heating output of the membrane heating 14b in dependence on the membrane temperature measured by the temperature sensor 14a and the membrane temperature preset via the temperature presetting means 16a.

The detector 20 is connected to an evaluation unit 22 receiving and evaluating the measuring signal of the detector 20. Using the evaluation device 22, the measuring values of the measuring signal generated by the detector 20 are acquired and the differences between measuring values are calculated.

In FIG. 2, the measuring signal S of the detector 20 is plotted as a function of time t in seconds. At the time t=0, the heating 14 is activated via the control device 16 and a first parameter P₁ is preset which causes the heating 14 to heat the membrane 12 to a first membrane temperature T₁ higher than the room temperature in the environment of the gas detection device 10. For example, the temperature T₁ may be 80° C. The temperature T₁ is the temperature at which the membrane 12 has a comparatively high permeability to the gas to be detected, which is helium in the present embodiment.

As a consequence, the measuring signal S increases, e.g. in the form of an electrical measuring current as illustrated in FIG. 2, to a maximum value that is measured as the first measuring value Hₙ at a time t of about 10 seconds. Thereafter, the parameter of the heating device 14 is changed from P₁ to P₂ via the control device 16, which results in the membrane temperature falling from the value T₁ to the value T₂. The second membrane temperature T₂ is lower than the first membrane temperature T₁, but is still higher than the room temperature in the environment of the gas detection device 10. In particular, the second membrane temperature T₂ is a temperature at which the permeability of the membrane 12 to the gas to be detected is lower than at T₁.

Therefore, the measuring signal S decreases from its local maximum due to the temperature T₁ to a local minimum due to the temperature T₂ and is measured as the second measuring value Lₙ at a time t of approximately 20 seconds. Subsequently, the parameter is changed from P₂ back to P₁ via the control device 16 and a further cycle of the periodic repetition of the temperature change is started thereby. The resultant first interval is identified as n in FIG. 2 and extends from 0 to a time t of 20 seconds. At the time t of 20 seconds, the next interval n+1 of the periodic repetition of the temperature switching starts. Changing the parameter of the control device 16 from P₂ to P₁ results in an increase of the membrane temperature up to the first membrane temperature T₁. At a time of about 30 seconds, the first measuring value of the second interval n+1 is then measured as Hₙ₊₁, before the parameter is again changed to P₂ and, as a result, the membrane temperature is lowered to T₂. The resultant measuring signal is measured as the second measuring value Lₙ₊₁ of the second interval n+1. The second interval then ends at 40 seconds and the third interval n+2 starts.

A linear drift of the offset of the measuring signal can be eliminated in the following manner:

The first measuring values Hₙ, Hₙ₊₁, Hₙ₊₂ etc. and the second measuring values Lₙ, Lₙ₊₁ etc. of the respective intervals n, n+1, n+2 etc. are acquired by the evaluation device 22. The evaluation device 22 calculates a difference between the first measuring value and the second measuring value, wherein, in the present embodiment, the mean value (Hₙ+Hₙ₊₁)/2 is calculated from the first measuring values Hₙ, Hₙ₊₁ of subsequent intervals n, n+1, and the second measuring value Lₙ acquired between the two first measuring values Hₙ, Hₙ₊₁ is subtracted therefrom. From this, the differential signal ΔSₙ of an interval n, with n being a natural number greater than 0, results as: ΔSₙ=(Hₙ+Hₙ₊₁)/2−Lₙ.

As an alternative, it is also conceivable that the mean value calculation can be performed using the second measuring values Lₙ, Lₙ₊₁ of two successive intervals n, n+1 as (Lₙ+Lₙ₊₁)/2, wherein the first measuring value is used in calculating the first measuring value Hₙ₊₁ that has been acquired between the two second measuring values Lₙ, Lₙ₊₁. Here, the resultant signal difference is

ΔSₙ=Hₙ₊₁(Lₙ+Lₙ₊₁)/2.

Basically, it is also conceivable that the mean values of both the first and the second measuring value can be calculated, wherein it is possible that the calculation of the mean values occurs over two or more intervals, respectively.

The second measuring value Lₙ, Lₙ₊₁ is acquired in each interval at a time at which the membrane temperature has been set to the second temperature T₂ and thereby the permeability of the membrane 12 is minimal. Therefore, the second measuring value Lₙ, Lₙ₊₁ respectively corresponds approximately to the offset signal which does not result from a gas that has passed the membrane 12.

Based on the differential signal A Sn, the evaluation unit 22 judges, whether the measuring signal S results from a gas to be detected and in what amount the gas is present, since the influence of the offset signal on the differential signal is reduced.

The control of the membrane temperature via the control device 16 and the heating 14 is effected by first measuring the membrane temperature using a measuring device not illustrated in the Figures. Depending on the temperature measurement result, the control device 16 causes the membrane 12 to be heated using the heating 14, until the higher, desired temperature value is reached.

Thus, the invention is based on the principle of a periodic change of the membrane temperature, in order to differentiate the offset signal from a useful signal resulting from a detected gas. Thereby, the amount of gas which, with the partial pressure difference of the gas pressures in front of and behind the membrane being preset, passes the membrane is changed periodically. The signal difference in the measuring signal between two temperature levels T₁, T₂ is proportional to the partial pressure difference and independent of the sensor offset. When the signal difference is measured and the system is calibrated as a result, the desired useful signal is obtained independently of the disturbing offset signal of the sensor.

The difference between the two temperature levels T₁, T₂ is only 5 K in the present embodiment. The signal deviation ΔS between the two temperature levels can be obtained by averaging of two adjacent signals of a high temperature, Hₙ+Hₙ₊₁, and the intermediate signal of the lower temperature level Lₙ. In principle, it is also possible to obtain the signal by averaging two low levels and the intermediate mean high level. In this manner, also a linear drift of the background is compensated for. The drift is visible in FIG. 2 as a negative gradient of the broken lines of the signal amplitudes falling from the left to the right.

The method of the present invention is used preferably for ambient air monitoring. Here, the sensor does not necessarily have to respond quickly. Likewise, no active conveyance of air is ultimately necessary.

The membrane temperature T₁, T₂ is varied in order to change the permeability of the membrane 12 and thus the sensor sensitivity of the gas detection device 10. This change of sensitivity is accompanied by a change of the measuring signal S, a change in current in the present case, which is proportional to the partial pressure of the gas to be detected (helium). The variation of the membrane temperature is achieved by changing the parameters P₁, P₂. Here, the parameters are set to values as low as possible, in order to generate a continuous sensor current (measuring signal S) that is as small as possible at atmospheric pressure, so that the service life of the sensor is not unnecessarily shortened. In the present embodiment in FIG. 2, the change of the parameter P₁, P₂ is effected every 10 seconds, switching back and forth between the parameters P₁ and P₂. After a change of the parameter, a period of approx. 8 seconds is awaited before the next measurement of the respective measuring value Hₙ, Lₙ in order to wait the period necessary for the control of the heating.

1.-13. (canceled)

14. A method for the detection of gas using a gas-selective membrane, a temperature device that is designed to change the temperature of the membrane, and a detector that is designed to detect a measurement signal on the basis of the amount of gas passing through the membrane,
characterized by
the following steps:
changing the temperature of the membrane using the temperature device,
acquiring at least one first measuring value using the detector at a time at which the membrane temperature adopts a first temperature value,
acquiring at least one second measuring value using the detector at a time at which the membrane temperature adopts a second temperature value different from the first temperature value,
calculating the difference between the two measuring values and
using the difference to assess whether a gas to be detected is present.

15. The method according to claim 14, wherein the change of the membrane temperature is performed periodically such that the membrane temperature alternately adopts the two temperature values at periodically recurring intervals, the measuring values for the respective temperatures being acquired during at least two different intervals.

16. The method according to claim 15, wherein the difference is calculated between the means value of the first measuring values of at least two different intervals and the second measuring value between the two first measuring values.

17. The method according to claim 15, wherein the difference is calculated between the means value of the second measuring values of at least two different intervals and the first measuring value between the two second measuring values.

18. The method according to claim 14, wherein changing the temperature is effected by measuring and controlling the temperature, the temperature control being performed between two temperature values greater than zero.

19. The method according to claim 14, wherein changing the temperature from one temperature value to the next temperature value is performed after at least 2 seconds and preferably after approx. at least 5-15 seconds.

20. The method according to claim 14, wherein the difference between the set temperature values of the membrane temperature is between approximately 2-10 K.

21. The method according to claim 14, wherein the difference between the set temperature values of the membrane temperature is between approximately 3-6 K.

22. The method according to claim 14, wherein the measuring values of the measuring signal are acquired in each interval only after at least approx. 2-5 seconds, after a new temperature value has been set for the temperature device.

23. The method according to claim 22, wherein the temperature device is a heating, at least the first temperature value of the membrane temperature being higher than the temperature of the environment of the membrane.

24. The method according to claim 23, wherein prior to setting a new temperature value of the membrane temperature, the heating is deactivated, the first temperature value and the second temperature value being each higher than the ambient temperature.

25. The method according to claim 14, wherein no pump is used to create a differential pressure between the pressures of the gas upstream of the membrane and the gas downstream of the membrane.

26. The method according to claim 14, wherein the method is used to detect a gas in a room of a building.

27. The method according to claim 14, wherein the detector is a gas measuring device or a pressure measuring device.
